(19) United States
(12) Patent Application Publication
(10) Pub. No.: US 2009/0306788 A1
(43) Pub. Date: Dec. 10, 2009

(54) IMPLANT FOR RESURFACING A FEMORAL HEAD

(76) Inventors: Michel Timoteo, Saint-Martin D ' Uriage (FR); Tarik Aitsi Selmi, Lyon (FR); Michel Bonnin, Lyon (FR)

Correspondence Address: YOUNG & THOMPSON, 209 Madison Street, Suite 500, ALEXANDRIA, VA, 22314, US

(21) Appl. No.: 12/282,390

(22) PCT Filed: Mar. 9, 2007
(86) PCT No.: PCT/FR07/00421

§ 371 Date: Jan. 26, 2009

Related U.S. Application Data
(60) Provisional application No. 60/812,948, filed on Jun. 13, 2006.

(30) Foreign Application Priority Data
Mar. 10, 2006 (FR) 0602126

(51) Int. Cl. A61F 2/36

(52) U.S. Cl. 623/23.14

(57) ABSTRACT

A resurfacing implant composed of a femoral cap (2) with an outer face (7), an inner face (8), a peripheral edge (11), a central axis XX′ passing through the pole P of the cap (2), an equatorial axis YY′ perpendicular to the first axis, and a point C of intersection between the main axes XX′, YY′ forming the centre of the radius of curvature of the outer (7) and inner (8) faces, characterized in that it includes guide elements composed of a cylindrical finger (9) with a base (90) connected to the inner face (8) at a point D, which is offset laterally with respect to the pole P of the hemispherical profile of the femoral cap (2), and stabilizing elements (10) arranged on the perimeter of the inner face (8), and having a profile arranged in a plane parallel to that containing the guide elements (9).

The present invention relates to a resurfacing implant for repairing the articular outer surface of the femoral head.

The surgical technique known as “resurfacing” for repairing the articular outer surface of the femoral head consists in substituting for the defective bony cartilage of the femoral head a hollow hemispherical metal cap. This femoral cap is generally associated with a metal cotyle which is also hemispherical and is driven into the acetabulum of the pelvis.

Note that current femoral caps have a globally cylindrical interior shape, obliging the surgeon to use cutting tools to work the originally spherical femoral head into a cylindrical shape.

This cutting of the femoral head can be considered invasive and contradictory to the basic thinking of this surgical technique, which by definition seeks to conserve the bone of the femoral head.

Also, the upper cortex of the femoral neck may be damaged by the cutting tool, with the risk of causing an onset of fracture.

It is therefore found that, after being cut by the surgeon to a cylindrical shape, the femoral head no longer has its cortex or layer of hard bone and has a cylindrical stump consisting essentially of weak spongy bone.

Note also that the profile of the above hemispherical cap imposes a unique position of said cap around the femoral head. This unique position is located on the axis of the femoral neck and can lead to tilting of the hemispherical cap through collapse of the bony support.

This surgical technique is therefore subject to certain drawbacks that are caused primarily by the disappearance of the bone of the femoral head and additionally by the lack of choice as to the position of the hemispherical cap as a function of the morphological state of the proximal end of the femur.

An object of the resurfacing implant of the present invention is to offer a solution to the problems set out above.

The resurfacing implant according to the present invention is composed of a femoral cap with an outer face, an inner face, a peripheral edge, a central axis XX′ passing through the pole P of said cap, an equatorial axis YY′ perpendicular to the first-mentioned axis, and a point C of intersection between the main axes XX′, YY′ forming the center of the radius of curvature of said outer and inner faces, guide means, which are composed of a cylindrical finger with a base connected to the inner face at a point D which is offset laterally with respect to the pole P of the hemispherical profile of the femoral cap, and stabilizing means disposed on the perimeter of the inner face and having a profile disposed in a plane parallel to that containing the guide means.

The resurfacing implant of the present invention has essential features that are defined and protected by claims 2 to 19.

The following description with reference to the appended drawings, provided by way of nonlimiting examples, explains the invention, its features, and the advantages that it offers:

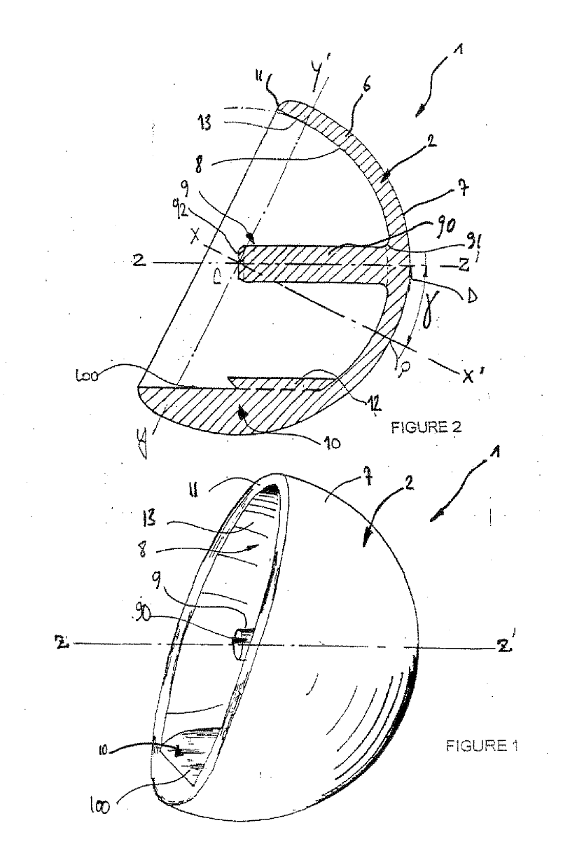

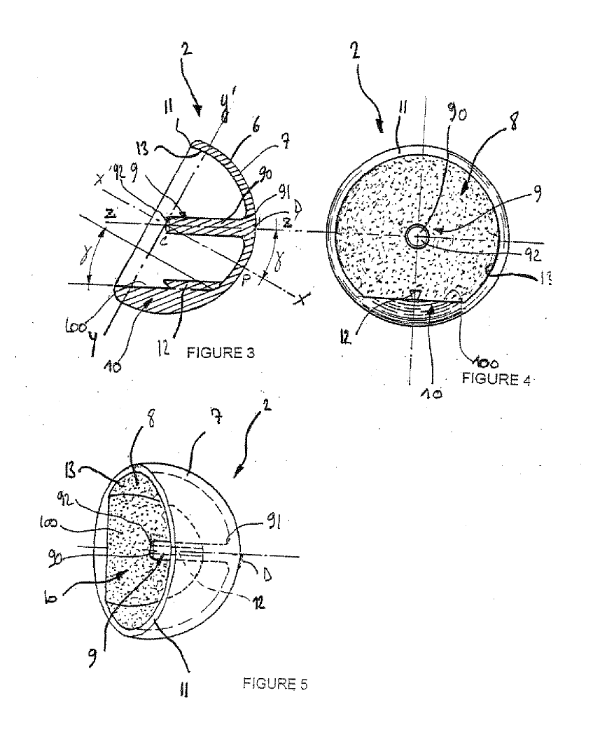

FIGS. 1 to 5 are views showing a resurfacing implant of the present invention.

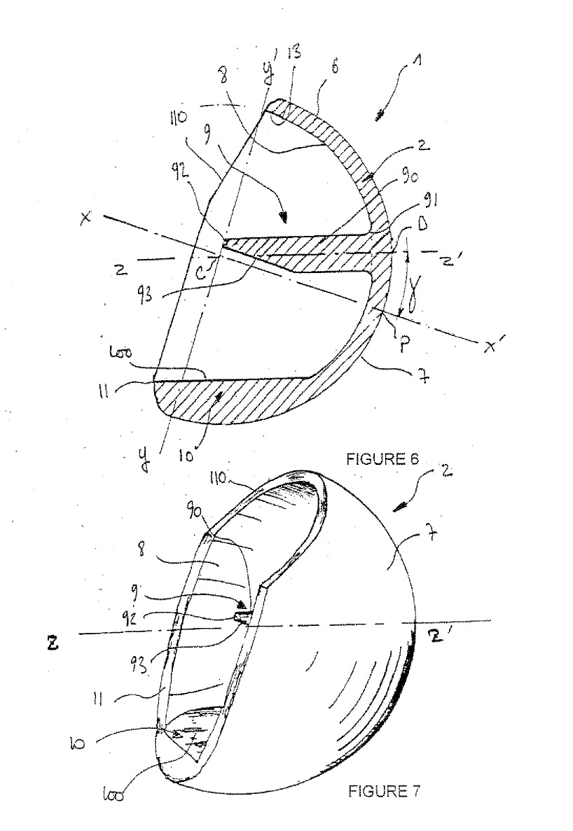

FIGS. 6 and 7 are views representing a first variant of the guide means and the stabilizing means of the resurfacing implant of the present invention.

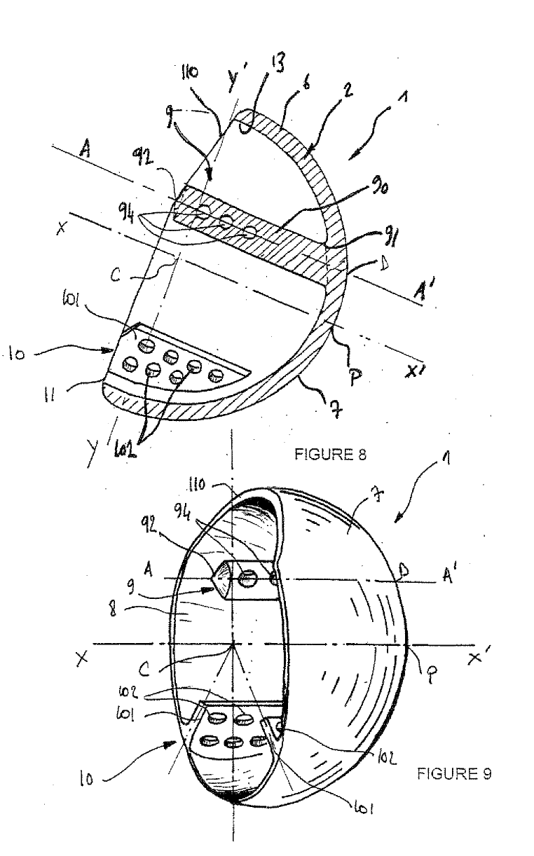

FIGS. 8 and 9 are views representing a second variant of the guide means and the stabilizing means of the resurfacing implant of the present invention.

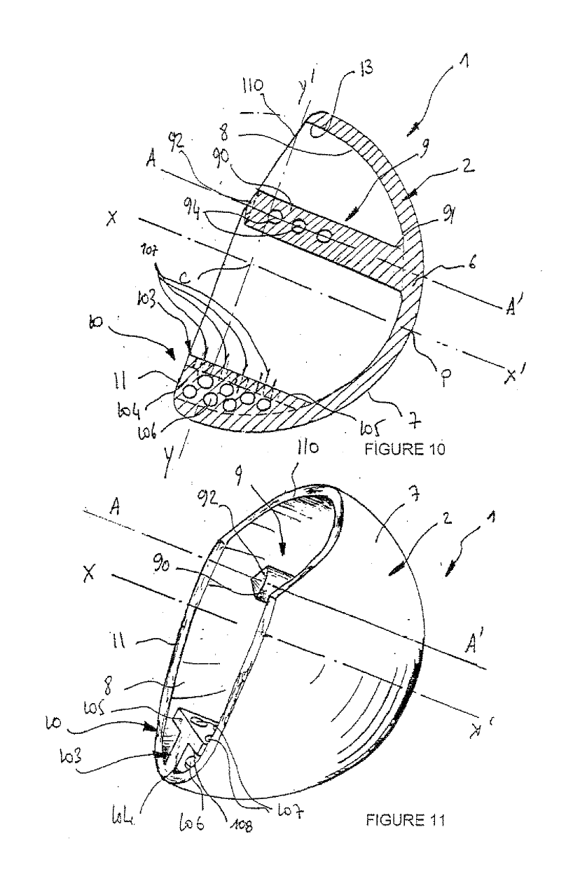

FIGS. 10 and 11 are views representing a third variant of the guide means and the stabilizing means of the resurfacing implant of the present invention.

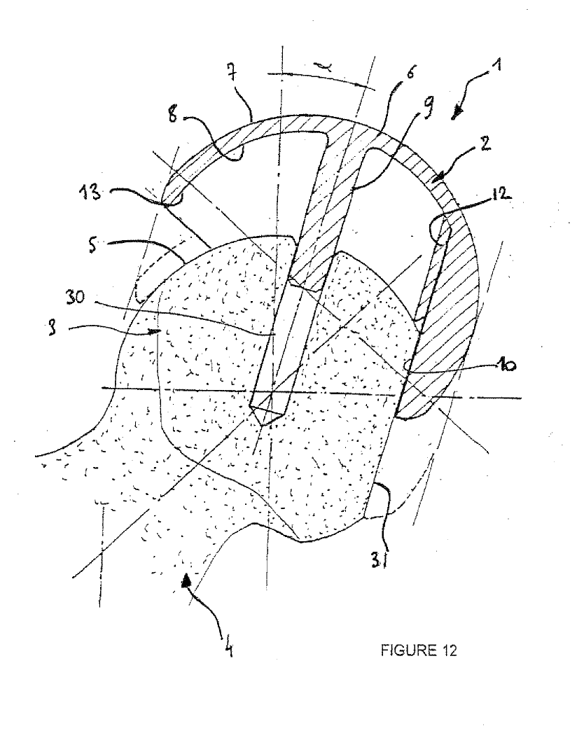

FIG. 12 is a view in section showing the fitting of the resurfacing implant to the femoral head.

FIGS. 1 to 5 show a resurfacing implant 1 of the present invention including a femoral cap 2 that is intended to be fixed to the femoral head 3 of a femur 4 the articular outer surface 5 whereof has deteriorated.

In order to be able to adapt to the dimensions of the femoral head 3 to be repaired, the resurfacing implant 1 is composed of a set of femoral caps 2.

The femoral caps 2 are composed of a thin wall 6 of constant thickness throughout the range and having a sliding outer face 7 of substantially hemispherical profile and an inner face 8 of partially hemispherical profile.

The femoral cap 2 has main axes composed of a central axis XX′ passing through the pole P of said cap 2 and an equatorial axis YY′ perpendicular to the first-mentioned axis and intersecting the central axis XX′ at a point C forming the center of the radius of curvature of the outer face 7 and the inner face 8.

The sliding outer face 7 has a spherical shape that extends beyond the equatorial axis YY′ as far as the peripheral edge 11 of the femoral cap 2. The sliding outer face 7 is also polished to a mirror finish.

The thickness of the wall 6 must not be greater than that of the articular outer surface 5 to be restored in order to conserve as much as possible of the bone of the femoral head and the greater part of its roundness.

The femoral cap 2 includes, upstanding from its inner face 8, guide means 9 that are composed of a cylindrical profile finger 90, which can be disposed on an axis ZZ′ passing through the center C.

The axis ZZ′ of the cylindrical finger 90 can be disposed in a direction inclined at an angle α relative to the central axis XX′ of the femoral cap 2.

The cylindrical finger 9 includes a base 91 joined to the inner face 8 at a point D on the axis ZZ′ and offset laterally relative to the pole P of the hemispherical profile of the femoral cap 2.

Also, the length of the cylindrical finger 90 is such that it does not extend beyond the peripheral edge 11 of the femoral cap 2, in order to be contained entirely within said cap.

To this end, the free end 92 of the cylindrical finger 90, at the end opposite the base 91, can be designed so as not to extend beyond the point C forming the center of the radius of curvature of the outer face 7 and the inner face 8 of the femoral cap 2.

The femoral cap 2 includes stabilizing means 10 that are disposed on the peripheral edge of the inner face 8 and feature at least one profile that is disposed in a plane parallel to that containing the guide means 90.

The inner face 8 of the femoral cap 2 includes stabilization means 10 that are composed of a truncated profile produced by at least one flat 100 that is disposed in a plane parallel to that containing the cylindrical finger 90.

The stabilizing means 10 can include, in the middle of the flat 100 and in the same plane as that containing the cylindrical finger 90, at least one tapered rib 12, for example having a dovetail profile.

In a nonlimiting preferred embodiment, the flat 100 includes a tapered rib 12 that extends from the inner face 8 of the femoral cap 2 and the length of which is substantially half that of said flat 100.

The inner face 8 includes a cylindrical inner face portion 13 disposed from the peripheral edge 11 of the femoral cap 2 to the equatorial axis YY′ of said femoral cap 2.

Note that the wall 6 of the femoral cap 2 increases in thickness at the location of the flat 100 formed in alignment with the inner face 8.

The inner face 8 is covered over the whole of its surface with an osteo-conductor substrate.

FIGS. 6 and 7 show a first variant of the guide means 9 and the stabilizing means 10 of the resurfacing implant 1 of the present invention.

The femoral cap 2 of the resurfacing implant 1 is similar to that described above with reference to FIGS. 1 to 5 and incorporates modifications concerning only the guide means 9 and the stabilizing means 10.

To this end, the femoral cap 2 includes, upstanding from its inner face 8, guide means 9 that are composed of a cylindrical profile finger 90, which can be disposed on an axis ZZ′ passing through the center C.

The axis ZZ′ of the cylindrical finger 90 can be disposed in a direction inclined at an angle α relative to the central axis XX′ of the femoral cap 2.

The cylindrical finger 90 includes a base 91 joined to the inner face 8 at a point D on the axis ZZ′ and offset laterally relative to the pole P of the hemispherical outer profile of the femoral cap 2.

Also, the length of the cylindrical finger 90 is such as not to extend beyond the peripheral edge 11 of the femoral cap 2 in order to be contained entirely within said cap.

To this end, the free end 92 of the cylindrical finger 90, at the end opposite the base 91, can be adapted not to extend beyond the point C forming the center of the radius of curvature of the outer face 7 and the inner face 8 of the femoral cap 2.

The cylindrical finger 90 has, extending from its free end 92 in the direction of the inner face 8 of the femoral cap 2, an inclined flat 93 producing a tapered profile of said cylindrical finger.

The inclined flat 93 can be disposed in a plane that is parallel to that containing the main axis XX′ passing between the point C and the pole P of the femoral cap 2.

The femoral cap 2 includes stabilizing means 10 that are disposed on the perimeter of the inner face 8 and feature at least one profile that is disposed in a plane parallel to that containing the guide means 9.

The inner face 8 of the femoral cap 2 includes stabilizing means 10 that are composed of a truncated profile produced by at least one flat 100 that is disposed in a plane parallel to that containing the cylindrical finger 90.

Note that in this case the flat 100 does not include any bone anchoring means as described with reference to FIGS. 1 to 5.

The inner face 8 includes a cylindrical inner face portion 13 disposed between the peripheral edge 11 of the femoral cap 2 and extending as far as the equatorial axis YY′ of said femoral cap 2.

For example, the peripheral edge 11 has, opposite the flat 100, an inclined sector 110 the lowest portion whereof is on or slightly below the equatorial axis YY′.

FIGS. 8 and 9 show a second variant of the guide means 9 and the stabilizing means 10 of the resurfacing implant 1 of the present invention.

The femoral cap 2 of the resurfacing implant 1 is similar to that described above with reference to FIGS. 1 to 5 and incorporates modifications concerning only the guide means 9 and the stabilizing means 10.

To this end, the femoral cap 2 includes, upstanding from its inner face 8, guide means 9 that are composed of a cylindrical profile finger 90 that can be disposed on an axis AA′ offset laterally relative to the axis XX′ but disposed in a plane parallel to that containing the main axis XX′ passing between the point C and the pole P of the femoral cap 2.

The cylindrical finger 90 includes a base 91 that is joined to the inner face 8 at a point D on the axis AA′ and offset laterally relative to the pole P of the hemispherical outer profile of the femoral cap 2.

Also, the length of the cylindrical finger 90 is such that it does not project beyond the peripheral edge 11 of the femoral cap 2 in order to be contained entirely within said cap.

To this end, the free end 92 of the cylindrical finger 90, at the end opposite the base 91, can be situated between the peripheral edge 11 of the femoral cap 2 and the equatorial axis YY′.

The cylindrical finger 90 incorporates at least one open hole 94 passing completely through the thickness of said finger to constitute an area facilitating bony rehabilitation.

For example, the open holes 94 are disposed on the one hand on the axis AA′ and on the other hand between the equatorial axis YY′ and the inner face 8 of the femoral cap 2.

The femoral cap 2 includes stabilizing means 10 that are disposed on the perimeter of the inner face 8 so that the guide means 9 and the stabilizing means 10 are disposed on each side of the main axis XX′ of the femoral cap 2.

The stabilizing means 10 are composed of at least one rib 101 extending from the inner face 8 in the direction of the main axis XX′ of the femoral cap 2.

Each rib 101 is composed of a thin wall that is anchored in the bony part of the femoral head 3 in order to stabilize the femoral cap and prevent any rotation thereof around the cylindrical finger 90.

Each rib 101 has open holes 102 formed completely through the thickness of the wall in order to constitute an area facilitating bony rehabilitation.

The inner face 8 includes a cylindrical inner face portion 13 disposed from the peripheral edge 11 of the femoral cap 2 as far as the equatorial axis YY′ of said femoral cap 2.

For example, the peripheral edge 11 has, opposite the stabilizing means 10, an inclined sector 110 the lowest portion of which is either on or slightly below the equatorial axis YY′.

FIGS. 10 and 11 show a third variant of the femoral cap 2 and more particularly of the stabilizing means 10 of the resurfacing implant 1 of the present invention, the guide means 9 being similar to those described above.

The stabilizing means 10 are composed of at least one rib 103 having a profile with three branches.

To this end, the rib 103 includes a first branch 104 extending from the inner face 8 in the direction of the main axis XX′ of the femoral cap 2, and two other branches 105, 108 are disposed in a direction that is perpendicular to that of the first branch so as to produce a T-shaped profile, for example.

Also, the branches 105, 108 can be disposed in a plane that is parallel to that containing the main axis XX′ of the femoral cap 2.

Each branch 104, 105 and 108 of the rib 103 consists of a thin wall that is anchored in the bony portion of the femoral head 3 in order to stabilize the femoral cap and prevent any rotation thereof about the cylindrical finger 90.

Each branch 104, 105 and 108 of the rib 103 is formed with open holes 106, 107 passing completely through the thickness of the wall in order to constitute an area facilitating bony rehabilitation.

FIG. 12 shows a femoral cap 2 that is positioned around the femoral head 3 so that on the one hand the cylindrical finger 90 enters a blind hole 30 and on the other hand the flat 100 cooperates with the plane face 31 obtained after cutting the articular outer surface 5.

Before fitting the femoral cap 2, the surgeon, with the aid of an ancillary, not shown, cuts the articular outer surface 5 of the femoral head 3 and offers up an appropriate femoral cap 2 selected from a set or range of caps.

Note that the cylindrical finger 90 is introduced into the blind hole 30 in the femoral head 3 before the tapered rib 12 comes into contact with the edge of the articular surface 5.

The surgeon then applies a pressure force to the femoral cap 2 so that it is driven into the femoral head 3 and covers the articular outer surface 5.

Note that on fixing the femoral cap 2 the tapered rib 12 enters the thickness of the femoral head 3, in the vicinity of the plane surface 31 formed when cutting the articular outer surface 5, to constitute an anchorage for said femoral cap 2 and to immobilize it against rotation about the cylindrical finger.

Note that the cylindrical finger 90 of the femoral cap 2, upon its introduction into the blind hole 30, serves primarily to guide the latter cap during its placement in order to obtain the most appropriate position possible.

Also, the flat 100 formed inside the femoral cap 2 comes to bear against the plane face 31 of the femoral head 3 to prevent said cap tilting on the roundness of said femoral head.

This fitting procedure is described by way of example and is specific to the femoral cap 2 described with reference to FIGS. 1 to 5.

The placement of the femoral cap 2 may be different, as a function of the structure of the guide means 9 and the stabilizing means 10 adopted.

Also, each variant described above is one embodiment of a femoral cap 2 and the combinations of the guide means 9 and the stabilizing means 10 proposed are not limiting on the invention and can be modified without changing the object of the present invention.

It must also be understood that the foregoing description has been given by way of example only and that it is in no way limiting on the scope of the invention, and that replacing the implementation details described by any other equivalent details does not depart from the scope of the invention.

1. A resurfacing implant composed of a femoral cap (2) with an outer face (7), an inner face (8), a peripheral edge (11), a central axis XX′ passing through the pole P of said cap (2), an equatorial axis YY′ perpendicular to the first-mentioned axis, and a point C of intersection between the main axes XX′, YY′ forming the center of the radius of curvature of said outer face (7) and said inner face (8), characterized in that it comprises guide means (9) composed of a cylindrical finger (90) with a base (91) connected to the inner face (8) at a point D which is offset laterally with respect to the pole P of the hemispherical profile of the femoral cap (2), and stabilizing means (10) disposed on the perimeter of the inner face (8) and having a profile disposed in a plane parallel to that containing the guide means (9).

2. The resurfacing implant as claimed in claim 1, characterized in that the cylindrical finger (90) includes, at the end opposite the base (91), a free end (92) that is located at the point C forming the center of the radius of curvature of the outer face (7) and the inner face (8) of the femoral cap (2).

3. The resurfacing implant as claimed in claim 1, characterized in that the cylindrical finger (90) includes, at the end opposite the base (91), a free end (92) that is situated between the peripheral edge (11) and the equatorial axis YY′ of the femoral cap (2).

4. The resurfacing implant as claimed in claim 1, characterized in that the cylindrical finger (90) includes an inclined flat (93) for producing a tapered profile.

5. The resurfacing implant as claimed in claim 1, characterized in that the cylindrical finger (90) is formed with at least one open hole (94) passing completely through the thickness of said finger in order to constitute an area facilitating bony rehabilitation.

6. The resurfacing implant as claimed in claim 1, characterized in that the cylindrical finger (90) includes an inclined flat (93) that is disposed in a plane that is parallel to that containing the main axis XX′ of the femoral cap 2.

7. The resurfacing implant as claimed in claim 1, characterized in that the stabilizing means (10) consist of a truncated profile produced by at least one flat (100).

8. The resurfacing implant as claimed in claim 7, characterized in that the flat (100) includes a tapered rib (12) that extends from the inner face (8) of the femoral cap (2).

9. The resurfacing implant as claimed in claim 7, characterized in that the flat (100) is disposed in a plane that is parallel to that containing the cylindrical finger (90).

10. The resurfacing implant as claimed in claim 1, characterized in that the stabilizing means (10) are composed of at least one rib (101) extending from the inner face (8) and in the direction of the main axis XX′ of the femoral cap (2).

11. The resurfacing implant as claimed in claim 10, characterized in that each rib (101) is formed with open holes (102) in order to constitute an area facilitating bony rehabilitation.

12. The resurfacing implant as claimed in claim 1, characterized in that the stabilizing means (10) are composed of at least one rib (103) having a profile with three branches.

13. The resurfacing implant as claimed in claim 12, characterized in that the rib (103) includes a first branch (104) extending from the inner face (8) in the direction of the main axis XX′ of the femoral cap (2) and two other branches (105, 108) disposed in a direction that is perpendicular to that of the first branch so as to produce a T-shaped profile.

14. The resurfacing implant as claimed in claim 13, characterized in that each branch (104, 105, 108) of the rib (103) is formed with open holes (106, 107) in order to constitute an area facilitating bony rehabilitation.

15. The resurfacing implant as claimed in claim 1, characterized in that the cylindrical finger (90) is disposed on an axis ZZ′ passing through the center C.

16. The resurfacing implant as claimed in claim 15, characterized in that the axis ZZ′ of the cylindrical finger (90) is disposed in a direction inclined at an angle α relative to the main axis XX′ of the femoral cap (2).

17. The resurfacing implant as claimed in claim 15, characterized in that the cylindrical finger 90 is disposed on an axis AA′ offset laterally and parallel to the main axis XX′ of the femoral cap 2.

18. The resurfacing implant as claimed in claim 15, characterized in that the peripheral edge (11) includes an inclined sector (110).

19. The resurfacing implant as claimed in claim 1, characterized in that the inner face (8) includes a cylindrical inner face portion (13) disposed from the peripheral edge (11) of the femoral cap (2) as far as the equatorial axis YY′ of said femoral cap.
